SEQUENCE LISTING (14 sequences)

SEQ ID NO: 1 - DNA, 20 bp, Artificial Sequence
  PTP1B forward primer
aagacccatcttccgtggac

SEQ ID NO: 2 - DNA, 20 bp, Artificial Sequence
  PTP1B reverse primer
acagacgcctgagcactttg

SEQ ID NO: 3 - DNA, 20 bp, Artificial Sequence
  TC-PTP forward primer
gctggcagccgttatacttg

SEQ ID NO: 4 - DNA, 20 bp, Artificial Sequence
  TC-PTP reverse primer
tggccaggtggtataatgga

SEQ ID NO: 5 - DNA, 18 bp, Artificial Sequence
  SHP2 forward primer
tggtttcaccccaacatc

SEQ ID NO: 6 - DNA, 20 bp, Artificial Sequence
  SHP2 reverse primer
cgtgggtcactttggacttg

SEQ ID NO: 7 - DNA, 20 bp, Artificial Sequence
  MEG2 forward primer
cctggaatgtggctgtcaag

SEQ ID NO: 8 - DNA, 20 bp, Artificial Sequence
  MEG2 reverse primer
atgctcccttcagcaggttt

SEQ ID NO: 9 - DNA, 20 bp, Artificial Sequence
  LYP forward primer
ttcctgaacaaagcctcacg

SEQ ID NO: 10 - DNA, 20 bp, Artificial Sequence
  LYP reverse primer
gggagttgatttggtccgtt

SEQ ID NO: 11 - DNA, 18 bp, Artificial Sequence
  RPTP beta forward primer
agatcaagggtgggcatt

SEQ ID NO: 12 - DNA, 20 bp, Artificial Sequence
  RPTP beta reverse primer
atgggactatccggatttgg

SEQ ID NO: 13 - DNA, 20 bp, Artificial Sequence
  GAPDH forward primer
accacagtccatgccatcac

SEQ ID NO: 14 - DNA, 20 bp, Artificial Sequence
  GAPDH reverse primer
tccaccaccctgttgctgta

(19) United States
(12) Patent Application Publication
(10) Pub. No.: US 2018/0325925 A1
(43) Pub. Date: Nov. 15, 2018

(54) COMPOSITION FOR PREVENTION OR TREATMENT OF NEUROINFLAMMATORY DISEASE, CONTAINING PROTEIN TYROSINE PHOSPHATASE INHIBITOR

(71) Applicants: KYUNGPOOK NATIONAL UNIVERSITY INDUSTRY-ACADEMIC COOPERATION FOUNDATION, Daegu (KR); KYUNGPOOK NATIONAL UNIVERSITY HOSPITAL, Daegu (KR)

(72) Inventors: Kyoung Ho Suk, Daegu (KR); Gyun Jee Song, Daegu (KR)

(21) Appl. No.: 15/773,928

(22) PCT Filed: Nov. 7, 2016
(86) PCT No.: PCT/KR2016/012746

§ 371 Date: Jul. 31, 2018

(30) Foreign Application Priority Data
Nov. 6, 2015 (KR) 10-2015-0155961
Nov. 7, 2016 (KR) 10-2016-0147537

(51) Int. Cl. A61K 31/662; A61P 25/00; A61P 29/00; A61K 9/00

(52) U.S. Cl. CPC A61K 31/662; A61K 9/0056; A61P 29/00; A61P 25/00

(57) ABSTRACT

The present invention relates to a pharmaceutical composition for prevention or treatment of a neuroinflammatory disease, in which the pharmaceutical composition includes a protein tyrosine phosphatase inhibitor. The protein tyrosine phosphatase inhibitor of the present invention inhibits activated microglia by decreasing the level of nitric oxide (NO) in the microglia and reducing the expression of proinflammatory factors, such as TNFα, IL1β, and iNOS, thereby being favorably used in prevention or treatment of the neuroinflammatory disease.

### TECHNICAL FIELD

The present invention relates to a pharmaceutical composition for prevention or treatment of a neuroinflammatory disease, the pharmaceutical composition including a protein tyrosine phosphatase inhibitor.

### BACKGROUND ART

The central nervous system consists of neurons and glial cells. The glial cells account for about 90% of total brain cells, and the volume accounts for about 50% of the entire brain. The glial cells can again be classified into three types: astrocytes, microglia, and oligodendrocytes. Among these, microglia are a type of specialized macrophages that are widely distributed in the brain. Microglia not only act as phagocytic cells that swallow up tissue debris and dead cells, but also play a part in biodefense activities of the brain.

Neuroinflammation is a type of immune response in the nervous system that is strongly associated with many neurodegenerative diseases including Alzheimer's disease, Parkinson's disease, and multiple sclerosis, and is now considered to be a hallmark of neurodegenerative diseases. The neuroinflammatory responses include the activation of innate immune cells (microglia), inflammatory mediators such as nitric oxide (NO), release of cytokines and chemokines, macrophage infiltration, which leads to neuronal cell death. The inflammation activation of the microglia and astrocytes is considered to be an important mechanism underlying the progression of pathologic markers and neurodegenerative diseases. Tight control of microglial activation is essential for maintaining brain homeostasis and preventing infection and inflammatory diseases.

On the other hand, protein tyrosine phosphatase (hereinafter referred to as ‘PTP’) is a group of enzymes that remove phosphate groups from tyrosine residues of phosphorylated proteins. Protein tyrosine phosphorylation is a common post-translational modification, generating new recognition motifs for protein interactions, affecting protein stability, and regulating enzyme activity. Thus, maintaining adequate levels of protein tyrosine phosphorylation is essential for cell function. A variety of proteins are known as protein tyrosine phosphatases. Among them, PTP1B (protein tyrosine phosphatase 1B) is one of the protein tyrosine dephosphorylases and is a major negative regulator of the insulin and leptin signaling pathways. In a mouse study in which PTP1B was removed, it was confirmed that PTP1B detected insulin, and PTP1B inhibitors exhibited protective effects against diabetes. Many studies have shown that PTP1B is associated with cancer, but currently there is little known about the relationship between protein tyrosine dephosphorylase and neuroinflammation.

Although many studies have been made to treat neuroinflammatory diseases, since the effects have not yet been clearly demonstrated and commercialized to be applicable to a wide range of neuroinflammatory diseases, there is a need to study new therapeutic agents.

### DISCLOSURE

### Technical Problem

Accordingly, the inventors of the present invention have continued to investigate substances capable of fundamentally treating a wide range of neuroinflammatory diseases by variously inhibiting activation of microglia and neuroinflammatory responses, and as a result, it has been confirmed that PTP inhibitors have an effect of inhibiting neuronal inflammation, and thus completing the present invention.

It is, therefore, an object of the present invention to provide a pharmaceutical composition for prevention or treatment of a neuroinflammatory disease, in which the pharmaceutical composition includes a protein tyrosine phosphatase inhibitor.

It is still another object of the present invention to provide a food composition for improving neuroinflammatory diseases, in which the food composition includes a PTP inhibitor.

It is another object of the present invention to provide a method of preventing or treating neuroinflammatory diseases, in which the method includes administering a PTP inhibitor to a subject.

### Technical Solution

In order to achieve the above object, the present invention provides a pharmaceutical composition for preventing or treating neuroinflammatory diseases, in which the pharmaceutical composition includes a PTP (protein tyrosine phosphatase) inhibitor.

The present invention also provides a food composition for improving neuroinflammatory diseases, in which the food composition includes a PTP inhibitor.

The present invention also provides a method of preventing or treating neuroinflammatory diseases, in which the method includes administering a PTP inhibitor to a subject.

### Advantageous Effects

The protein tyrosine dephosphorylase inhibitor of the present invention inhibits the activated microglia by decreasing the level of nitric oxide (NO) in the microglia, decreasing the expression of proinflammatory factors TNFα, IL1 β, iNOS, and the like, and thus can be used usefully for the prevention or treatment of neuroinflammatory diseases.

### DESCRIPTION OF DRAWINGS

FIG. 1 is a diagram illustrating the results of RT-PCR analysis of the expression level change of PTP mRNA by LPS in a mouse brain.

FIG. 2 is a diagram illustrating the results of RT-PCR analysis of the expression level change of PTP mRNA by LPS in mouse primary microglia and primary astrocytes.

FIG. 3 is a graph illustrating the results of confirming whether the PTP inhibitor inhibits the activation of microglial cell line BV2 and cytotoxicity by measurement of NO production level and MTT analysis.

FIG. 4 is a diagram illustrating the results of confirming whether the PTP inhibitor inhibits microglial activation. FIG. 4A illustrates an experimental design, FIG. 4B illustrates a histochemical examination of the morphological changes of the brain of mice, FIG. 4C illustrates the results of confirming Iba-1 positive cells, which are the microglial marker in the hippocampus, cortex, and thalamus of mice, and FIG. 4D is a graph quantifying the results of confirming Iba-1 positive cells, which are the microglial marker in the hippocampus, cortex, and thalamus of mice.

FIG. 5 is a diagram illustrating the results of confirming the expression level of PTP1B in the BV2 microglial cell line (HA-PTP1B) overexpressing the PTP1B produced.

FIG. 6 is a graph illustrating the result of measurement of NO production level in a microglial cell line BV2 stimulated by LPS according to the capacity of LPS.

FIG. 7 is a graph illustrating the results of measurement of NO production level in a microglial cell line BV2 stimulated by LPS over time.

FIG. 8 is a graph illustrating the results confirmed with real-time PCR of the expression level change of inflammatory cytokine mRNA in a BV2 microglial cell line overexpressing PTP1B.

FIG. 9 is a graph illustrating the results of confirming the expression level change of LPS-induced NO by treatment with PTP1B inhibitor (iPTP1B) in a microglial cell line BV2.

FIG. 10 is a graph illustrating the results of confirming the expression level change of LPS-induced NO by treatment with PTP1B inhibitor (iPTP1B) in primary microglia.

FIG. 11 is a graph illustrating the results of confirming the expression level change of LPS-induced NO by treatment with PTP1B inhibitor (iPTP1B) in rat microglia.

FIG. 12 is a graph illustrating the results of confirming the expression level change of TNFα-induced NO by treatment with PTP1B inhibitor (iPTP1B) in rat microglia.

FIG. 13 is a diagram illustrating the results of RT-PCR of the expression level change of proinflammatory molecules according to the treatment with PTP1B inhibitor (iPTP1B) in a microglial cell line BV2.

FIG. 14 is a graph illustrating the results of quantitative analysis of the expression level change of proinflammatory molecules by treatment with PTP1B inhibitor (iPTP1B) in a microglial cell line BV2 by RT-PCR.

FIG. 15 is a graph illustrating the results of the expression level change of TNFα protein according to the treatment with PTP1B inhibitor (iPTP1B) in a microglial cell line BV2 through ELISA.

FIG. 16 is a diagram confirming that the phosphorylation at the position of Src at Y527 is decreased by PTP1B in a microglial cell line BV2.

FIG. 17 is a graph confirming that the level of NO induced by LPS is increased in a microglial cell line overexpressing PTP1B.

FIG. 18 is a graph confirming that the level of NO induced by LPS is decreased by Src kinase inhibitor in microglia.

FIG. 19 is a graph illustrating the results of confirming that the treatment of the Src kinase inhibitor eliminates the anti-inflammatory effect of the PTP1B inhibitor.

FIG. 20 is a diagram illustrating the results of confirming that the PTP1B inhibitor increases the phosphorylation of Src at an Y527 position.

FIG. 21 is a schematic representation of the mechanism by which PTP1B is involved in neuroinflammation.

FIG. 22 is a diagram illustrating an experimental design for confirming the neuroinflammation mitigating effect of a PTP1B inhibitor in vivo.

FIG. 23 is a graph illustrating the results of confirming that a PTP1B inhibitor in vivo inhibits the expression of TNFα and IL1β by real-time PCR.

### MODES OF THE INVENTION

Hereinafter, the present invention will be described in detail.

The present invention provides a pharmaceutical composition for prevention or treatment of a neuroinflammatory disease, in which the pharmaceutical composition includes a PTP inhibitor.

In the present invention, “a PTP inhibitor (protein tyrosine phosphatase inhibitor)” includes inhibitors of one or more PTPs selected from the group consisting of PTP1B (Protein tyrosine phosphatase type 1B), TC-PTP (T-cell phosphatase), SHP2 (Src homology domain2-containing PTP2), MEG2 (Megakaryocyte-PTP2), LYP (Lymphoid specific-tyrosine phosphatase) and RPTPβ (Receptor-type tyrosine protein phosphatase beta), but is not limited thereto.

Such PTP inhibitors include, but are not limited to, the compounds listed in the following table or pharmaceutically acceptable salts thereof.

| Names of Compounds | Target PTP |
|---|---|
| (S)-4-(((S)-1- | PTP1B |
| (l2-azanyl)-3-(4-(difluoro(phosphono)methyl)phenyl)-1-oxopropan- | |
| 2-yl)amino)-3-((S)-3-(4-(difluoro(phosphono)methyl)phenyl)-2- | |
| pentadecanamidopropanamido)-4-oxobutanoic acid | |
| ((4-((S)-3-(((S)-1-amino-6-(4-ethylbenzamido)- | TC-PTP |
| 1-oxohexan-2-yl)amino)-2-((S)-2-(2-(((1R,2R,5S)-2-isopropyl- | |
| 5-methylcyclohexyl)oxy)acetamido)-3-phenylpropanamido)-3- | |
| oxopropyl)phenyl)difluoromethyl)phosphonic acid | |
| ((4- | SHP2 |
| ((S)-3-(((S)-1-(((S)-1-amino-3-(2-(4-hydroxy-3-methoxyphenyl)acetamido)- | |
| 1-oxopropan-2-yl)amino)-5-(3-iodobenzamido)-1-oxopentan-2- | |
| yl)amino)-6-hydroxy-3-iodo-1-methyl-2-(3-(2-oxo-2-((4-thiophen-3- | |
| yl)phenyl)amino)acetamido)phenyl)-1H-indole-5-carboxylic acid | |
| ((4-((S)-3-(((S)-1-(((S)-1-amino-3-(2-(4-hydroxy- | MEG2 |
| 3-methoxyphenyl)acetamido)-1-oxopropan-2-yl)amino)-5-(3-iodobenzamido)- | |
| 1-oxopentan-2-yl)amino)-2-(3-bromo-4-methylbenzamido)- | |
| 3-oxopropyl)phenyl)difluoromethyl)phosphonic acid | |
| 3-((3-Chlorophenyl)ethynyl)- | LYP |
| 2-(4-(2-(cyclopropylamino)-2-oxoethoxy)phenyl)-6-hydroxybenzofuran- | |
| 5-carboxylic Acid | |
| 2-(3-(2-(3-bromo-5-iodobenzamido)acetamido)phenyl)- | RPTPβ |
| 6-hydroxy-3-iodo-1-methyl-1H-indole- | |
| 5-carboxylic acid | |

Some of the compounds listed in the above table are disclosed in the prior art entitled Cellular Effects of Small Molecule PTP1B Inhibitors on Insulin Signaling (Biochemistry 2003, 42, 12792-12804), and it has not been revealed that the compounds can be used for the activity of inhibiting neuroinflammation and for the prevention or treatment of neuroinflammatory diseases accordingly.

In the present invention, the “pharmaceutically acceptable salt” is not limited as long as it forms an addition salt with the compounds, and includes salts derived from pharmaceutically acceptable inorganic acids, organic acids, or bases. Examples of suitable acid addition salts include acid addition salts formed by inorganic acids such as sulfuric acid, hydrochloric acid, nitric acid, phosphoric acid, bromic acid, perchloric acid, hydroiodic acid and the like; organic carboxylic acids such as oxalic acid, citric acid, succinic acid, tartaric acid, formic acid, acetic acid, trichloroacetic acid, trifluoroacetic acid, glycolic acid, benzoic acid, lactic acid, fumaric acid, maleic acid, salicylic acid and the like; and sulfonic acids such as methanesulfonic acid, ethanesulfonic acid, benzenesulfonic acid, toluene-p-sulfonic acid, naphthalene-2-sulfonic acid and the like. Examples of suitable base addition salts include base addition salts formed by alkali metal or alkaline earth metal salts formed by lithium, sodium, potassium, calcium, magnesium and the like; amino acid salts such as lysine, arginine and guanidine; and organic salts such as dicyclohexylamine, N-methyl-D-glucamine, tris(hydroxymethyl) methylamine, diethanolamine, choline, triethylamine, and the like.

The PTP1B inhibitor according to the present invention and the compound of formula 1 can be converted into a salt thereof by a conventional method, and the preparation of salts can be easily carried out by those skilled in the art based on the structure of the compound without any explanation.

The “neuroinflammatory diseases” in the present invention may include, without limitation, diseases caused by inflammation of the nervous system, such as multiple sclerosis, neuroblastoma, stroke, Alzheimer's disease, Parkinson's disease, Lou Gehrig's disease, Huntington's disease, Creutzfeldt-Jacob's disease, post traumatic stress disorder, depression, schizophrenia, and amyotrophic lateral sclerosis, but is not limited thereto.

The term “prevention” in the present invention refers to the inhibition of the development of an illness or a disease in a subject who has never been diagnosed as having a neuroinflammatory illness or disease, but tends to be susceptible to such illness or disease. In addition, the term “treatment” in the present invention refers to the inhibition of the development of neuroinflammatory illness or diseases, the alleviation of illness or diseases, and the elimination of illness or diseases.

In a specific embodiment of the present invention, it has been confirmed that the PTP inhibitor reduces the activation of microglia under inflammatory conditions induced by LPS (lipopolysaccharide), reduces the secretion of the inflammatory cytokines TNFα, IL1β and iNOS, and also reduces the production of nitric oxide (NO). Therefore, it has been confirmed that the pharmaceutical composition including a PTP inhibitor as an active ingredient can be usefully used for preventing or treating neuroinflammatory diseases by reducing the anti-inflammatory effect and the activation of microglia.

In a specific example of the present invention, the inhibitory effect of PTP inhibitor on neuroinflammation was verified through in vitro and in vivo experiments.

In a specific example of the present invention, it has been confirmed that PTP1B promotes the production of proinflammatory cytokines and activates Src through dephosphorylation of Src at an Y527 position, and that the Src activated as such also activates NFκB and increases the expression of proinflammatory factors. From these results, it was confirmed that an inhibitor of PTP1B can be usefully used as an active ingredient of a composition for preventing or treating neuroinflammatory diseases.

In addition, the pharmaceutical composition of the present invention may further include a pharmaceutically acceptable carrier. As used herein, the term “pharmaceutically acceptable carrier” refers to a carrier or diluent that does not significantly stimulate the organism and does not interfere with the biological activity and properties of the administered compound. Pharmaceutically acceptable carriers include, for example, carriers for oral administration such as lactose, starch, cellulose derivatives, magnesium stearate, stearic acid and the like, and carriers for parenteral administration such as water, suitable oils, saline solution, aqueous glucose, glycol and the like. Such a pharmaceutically acceptable carrier may be a mixture of saline solution, sterilized water, Ringer's solution, buffered saline solution, dextrose solution, maltodextrin solution, glycerol, ethanol and one or more components of these components. In addition, if necessary, other conventional additives such as stabilizers, preservatives, antioxidants, buffers and bacteriostatic agents may be added.

In addition, the pharmaceutical composition of the present invention can be prepared in various parenteral or oral administration forms according to known methods. Representative examples of formulations for parenteral administration include isotonic aqueous solutions or suspensions and the like as injection formulations, and can be prepared according to the technology known in the pertinent field by using suitable dispersing agents or wetting agents and suspending agents. For example, each component can be formulated into injection forms by being dissolved in saline solution or buffer solution. In addition, formulations for oral administration include, but are not limited to, powders, granules, tablets, pills, emulsions, syrups and capsules.

The present invention also provides a method of preventing or treating neuroinflammatory diseases, in which the method includes administering a PTP inhibitor to a subject.

The term “administration” as used herein refers to the introduction of the pharmaceutical composition of the present invention into a subject in need of treatment of a disease by any suitable method, and the administration route of the composition of the present invention can be administered through various routes of oral or parenteral administration as long as it can reach the target tissues.

The subject to be administered may be a mammal such as a rat, a mouse, a livestock, a human being, and may be administered through various routes including oral, transdermal, subcutaneous, intravenous, or intracerebral injection.

The prevention or treatment method of the present invention includes administering a pharmaceutically effective amount of a PTP inhibitor or a pharmaceutically acceptable salt thereof. It will be apparent to those skilled in the art that the appropriate total daily dose may be determined by a practitioner within the scope of sound medical judgment. For purposes of the present invention, it is preferable to differently apply the specific therapeutically effective amount for a particular patient depending upon a variety of factors, including the type and degree of response to be achieved, the specific composition, including whether other pharmaceutical preparations are used depending on cases, the age, weight, general health status, gender and diet of a patient, administration time, administration route and secretion rate of a composition, duration of treatment, drugs used together or simultaneously with the specific composition, and similar factors well known in the medical field.

The present invention also provides a food composition for improving neuroinflammatory diseases, in which the food composition includes a PTP inhibitor.

The term “food” as used herein refers to a natural product or a processed product including one or more nutrients, preferably a state of being able to be eaten directly through a certain degree of processing, and as an acceptable meaning, it is used to have a meaning of including all of various foods, health functional foods, beverages, food additives, and beverage additives.

The food composition of the present invention may be added to various foods, candy, chocolate, beverage, gum, tea, vitamin complex, various health supplement foods and the like, and may be used in the form of powders, granules, tablets, pills, capsules or beverages.

In addition, the food composition of the present invention may further include a sitologically acceptable carrier. There are no particular limitations other than those including the PTP inhibitor of the present invention or a sitologically acceptable salt thereof. For example, it may further include various flavoring agents or natural carbohydrates and the like. In addition, the food composition of the present invention includes components that are ordinarily added during the manufacture of foods, and may include, for example, proteins, carbohydrates, fats, nutrients, and seasonings. In addition, various nutritional supplements, vitamins, minerals, flavoring agents such as synthetic flavoring agents and natural flavoring agents, colorants, enhancers, factic acid and salts thereof, alginic acid and its salts, organic acid, protective colloid thickeners, pH regulators, stabilizers, preservatives, glycerin, alcohols, carbonating agents used in carbonated drinks, and the like may be included.

In addition, in terms of food, the amount of the PTP inhibitor or salt thereof may be 0.00001% to 50% by weight of the total food. When the food is a beverage, the amount of the PTP inhibitor or salt thereof may be included in a ratio of 0.001 g to 50 g, preferably 0.01 g to 10 g based on 100 ml by volume of the whole food, but is not limited thereto.

Terms not otherwise defined in the present specification have meanings as commonly used in the technical field to which the present invention pertains.

Hereinafter, the present invention will be described in detail with reference to examples and preparation examples. However, the following examples and preparation examples are merely illustrative of the present invention, and the content of the present invention is not limited to the following examples and preparation examples.

### Example 1. Identification of Expression Change of PTP Under Inflammatory Conditions

In order to confirm whether inflammatory conditions regulate the expression of PTP in mouse brains, mice injected with LPS were prepared as infectious animal models as follows.

1-1. Preparation of Neuroinflammatory Mouse Model

In order to induce neuroinflammation in mice, LPS (Lipopolysaccharide) was intraperitoneally administered. All experiments were performed using male C57BL/6 mice (25-30 g) of 9-11 weeks old, supplied by Koatech (Pyongtaec City, Korea), and mice were intraperitoneally injected with 5 mg/kg of LPS to prepare a neuroinflammatory mouse model. A vehicle was injected into a control group.

1-2. Identification of Expression of PTP in Mouse Brains Under Inflammatory Conditions

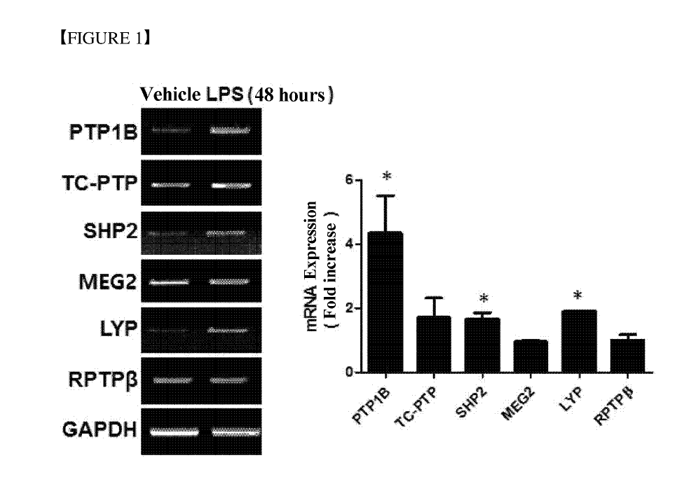

In order to confirm the role of PTP in mouse brains under inflammatory conditions, LPS was injected as in Example 1-1 above, and mRNA expression levels of PTP1B, TC-PTP, SHP2, MEG2, LYP, and RPTPβ were confirmed in the brain samples collected after 48 hours by RT-PCR. The primers are shown in the following Table 1 and the confirmation results are illustrated in FIG. 1.

TABLE 1

| | Number | | | | |
|---|---|---|---|---|---|
| Target | (Accession | | SEQ | | SEQ |
| gene | number) | Forward primer (5′−>3′) | ID NO. | Reverse primer (5′−>3′) | ID NO. |
| PTP1B | NM_011201.3 | AAGACCCATCTTCCGTGGAC | 1 | ACAGACGCCTGAGCACTTTG | 2 |
| TC-PTP | NM_008977.3 | GCTGGCAGCCGTTATACTTG | 3 | TGGCCAGGTGGTATAATGGA | 4 |
| SHP2 | NM_011202.3 | TGGTTTCACCCCAACATC | 5 | CGTGGGTCACTTTGGACTTG | 6 |
| MEG2 | NM_019651.2 | CCTGGAATGTGGCTGTCAAG | 7 | ATGCTCCCTTCAGCAGGTTT | 8 |
| LYP | NM_008979.2 | TTCCTGAACAAAGCCTCACG | 9 | GGGAGTTGATTTGGTCCGTT | 10 |
| RPTPβ | NM_001311064.1 | AGATCAAGGGTGGGCATT | 11 | ATGGGACTATCCGGATTTGG | 12 |
| GAPDH | NM_ 008084 | ACCACAGTCCATGCCATCAC | 13 | TCCACCACCCTGTTGCTGTA | 14 |

As illustrated in FIG. 1, it has been confirmed that mRNA expressions of PTP1B, TC-PTP, SHP2, and LYP were increased in the brain of inflammation-induced mice by injection of LPS. In particular, it has been confirmed that the expressions of PTP1B, SHP2, and LYP were significantly increased.

Therefore, the expression of PTP is increased in the brain of inflammation-induced mice by injection of LPS, indicating that the PTP is correlated with brain inflammation.

1-3. Identification of Expression of PTP in Mouse Primary Microglia and Primary Astrocytes Under Inflammatory Conditions

Microglia are immune cells present in the central nervous system and play a role in the initiation and progression of the inflammatory response resulting from inflammatory stimuli. In the primary microglia and primary astrocytes isolated from mice by RT-PCR method, the expression level change of PTP was confirmed by LPS.

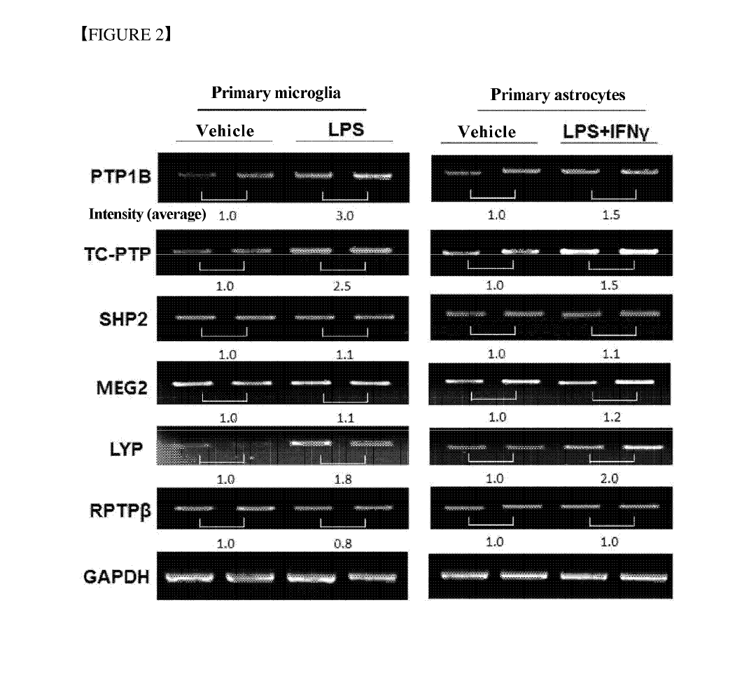

Specifically, mouse primary microglia were treated with LPS at 100 ng/ml and mouse primary astrocytes were treated with LPS and IFN-γ (10 U/ml) to induce an inflammatory response. mRNA levels of PTP1B, TC-PTP, SHP2, MEG2, LYP, and RPTPβ were confirmed by RT-PCR in primary microglia and primary astrocytes 24 hours after the treatment. The confirmation result is illustrated in FIG. 2.

As illustrated in FIG. 2, it has been confirmed that mRNA expressions of PTP1B, TC-PTP and LYP were increased.

Therefore, the expression of PTP is increased in the neuroglial cells under inflammatory conditions induced by the injection of LPS, indicating that PTP is associated with brain inflammation.

### Example 2. Identification of Inhibition of Microglial Activation of PTP Inhibitors

Since activated microglia induce neuroinflammatory responses by secretion of neurotoxic factors such as NO, NO production is a strong marker of inflammation response in microglia. In order to check whether PTP inhibitors inhibit microglial activation, the PTP inhibitors described in Table 2 below were used.

TABLE 2

| Names of Compounds | Target PTP |
|---|---|
| (S)-4-(((S)-1- | PTP1B |
| (l2-azanyl)-3-(4-(difluoro(phosphono)methyl)phenyl)-1-oxopropan- | |
| 2-yl)amino)-3-((S)-3-(4-(difluoro(phosphono)methyl)phenyl)-2- | |
| pentadecanamidopropanamido)-4-oxobutanoic acid | |
| ((4-((S)-3-(((S)-1-amino-6-(4-ethylbenzamido)- | TC-PTP |
| 1-oxohexan-2-yl)amino)-2-((S)-2-(2-(((1R,2R,5S)-2-isopropyl- | |
| 5-methylcyclohexyl)oxy)acetamido)-3-phenylpropanamido)-3- | |
| oxopropyl)phenyl)difluoromethyl)phosphonic acid | |
| ((4- | SHP2 |
| ((S)-3-(((S)-1-(((S)-1-amino-3-(2-(4-hydroxy-3-methoxyphenyl)acetamido)- | |
| 1-oxopropan-2-yl)amino)-5-(3-iodobenzamido)-1-oxopentan-2- | |
| yl)amino)-6-hydroxy-3-iodo-1-methyl-2-(3-(2-oxo-2-((4-thiophen-3- | |
| yl)phenyl)amino)acetamido)phenyl)-1H-indole-5-carboxylic acid | |
| ((4-((S)-3-(((S)-1-(((S)-1-amino-3-(2-(4-hydroxy- | MEG2 |
| 3-methoxyphenyl)acetamido)-1-oxopropan-2-yl)amino)-5-(3-iodobenzamido)- | |
| 1-oxopentan-2-yl)amino)-2-(3-bromo-4-methylbenzamido)- | |
| 3-oxopropyl)phenyl)difluoromethyl)phosphonic acid | |
| 3-((3-Chlorophenyl)ethynyl)- | LYP |
| 2-(4-(2-(cyclopropylamino)-2-oxoethoxy)phenyl)-6-hydroxybenzofuran- | |
| 5-carboxylic Acid | |
| 2-(3-(2-(3-bromo-5-iodobenzamido)acetamido)phenyl)- | RPTPβ |
| 6-hydroxy-3-iodo-1-methyl-1H-indole- | |
| 5-carboxylic acid | |

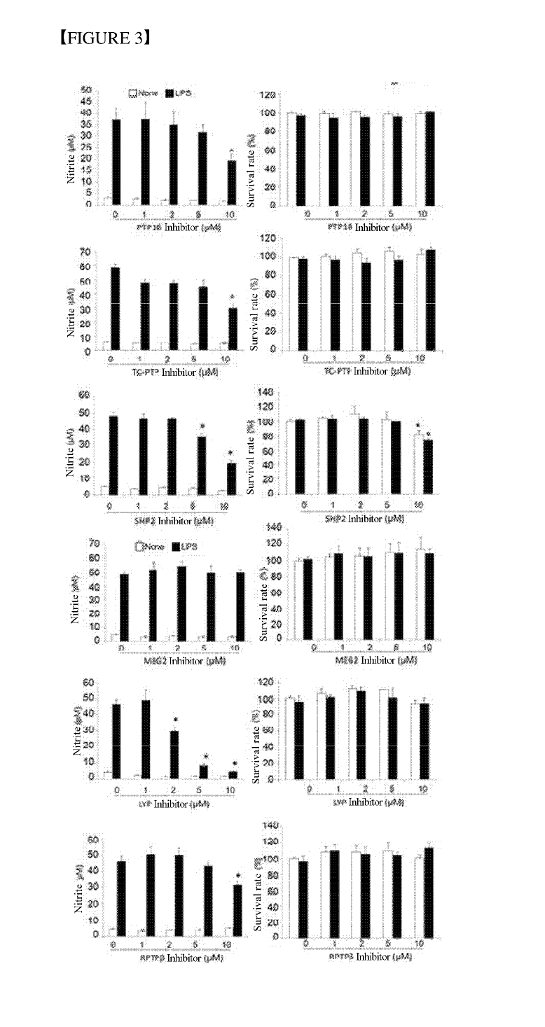

Specifically, BV-2 microglia were treated with LPS (100 ng/ml) for 24 hours in the presence of 1, 2, 5, and 10 μM of each PTP inhibitor, and the amount of NO was measured using the Greiss reaction. In addition, cytotoxicity was confirmed using MTT assay method, and the results are illustrated in FIG. 3.

As illustrated in FIG. 3, it was confirmed that the amount of NO induced by LPS decreased with the treatment of the PTP inhibitor, and it was confirmed that the cell survival rate was not significantly decreased.

Therefore, it was confirmed that the PTP inhibitor can inhibit the activation of microglia by safely reducing the amount of NO induced by LPS without cytotoxicity.

### Example 3. Identification of Inhibitory Effect of PTP Inhibitor on Microglial Activation in Brain Inflammation Model

3-1. Mouse Model

A brain inflammation mouse model was prepared to confirm whether PTP inhibitors inhibit microglial activation. C57BL/6 mice were injected intracerebrally with vehicle (saline solution containing 0.5% DMSO and 5% propylene glycol) or the PTP inhibitor of Table 2 above (dilution in saline solution containing 5% propylene glycol). LPS (5 mg/kg) was intraperitoneally injected 30 minutes after injection. Mice were sacrificed and the brain was analyzed 48 hours after LPS injection.

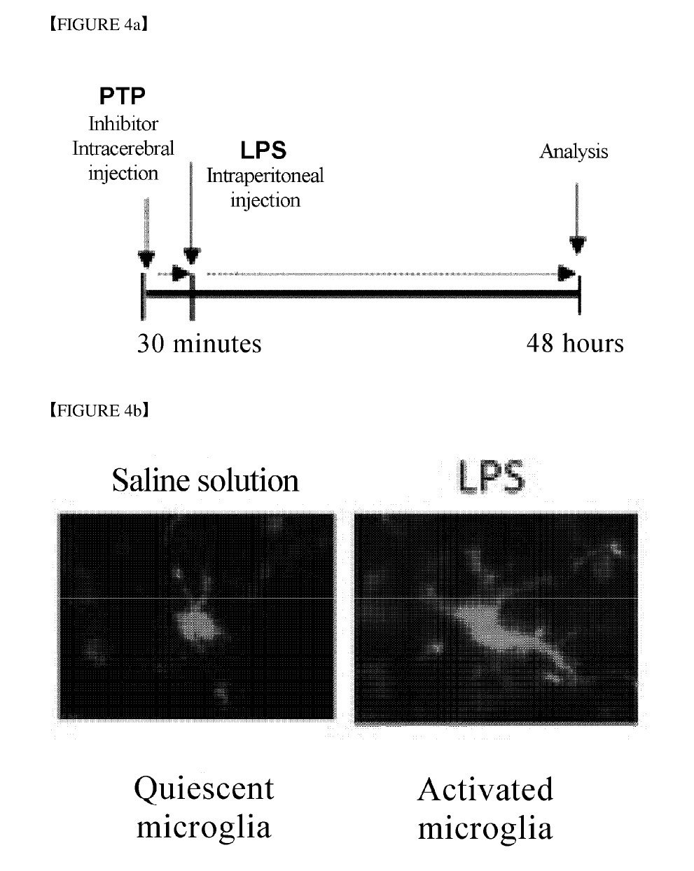

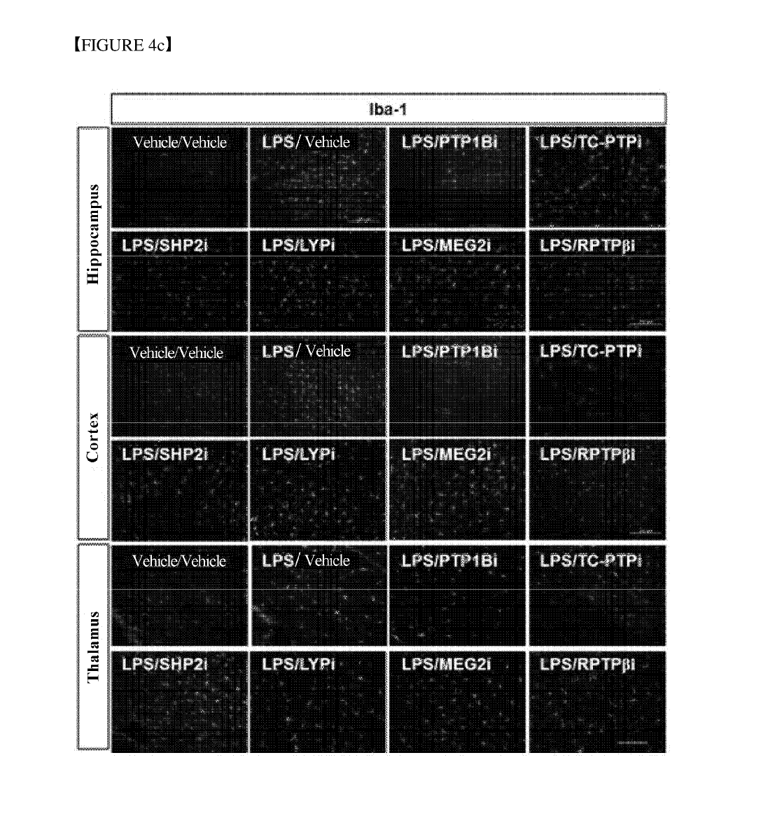

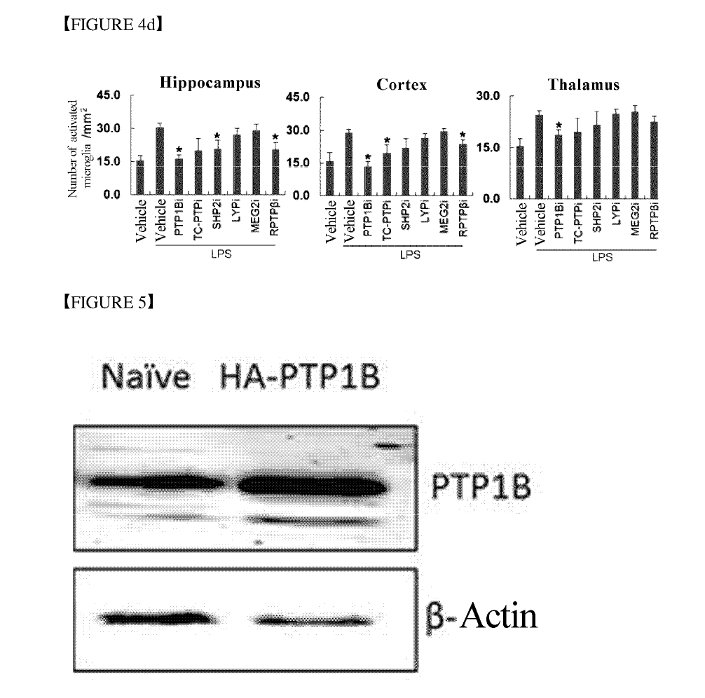

The mouse model was divided into eight experimental groups; Group 1 treated with saline solution and 0.5% DMSO; Group 2 treated with LPS and 0.5% DMSO; Group 3 treated with LPS and PTP1B inhibitor; Group 4 treated with LPS and TC-PTP inhibitor; Group 5 treated with LPS and SHP2 inhibitor; Group 6 treated with LPS and MEG2 inhibitor; Group 7 treated with LPS and LYP inhibitor; and Group 8 treated with LPS and RPTPβ inhibitor. The experimental design above is illustrated in FIG. 4A.

FIG. 4B illustrates the histochemical results of confirming whether the sacrificed mouse brain is morphologically changed by straining the antibody against Iba-1, a marker of microglia. As illustrated in FIG. 4B, morphological changes of microglia after injection of LPS were observed.

In addition, the brains were removed and sectioned, and the hippocampus, cortex and thalamus were stained with an antibody against Iba-1. The results of histochemical confirmation are illustrated in FIG. 4C. The results of graphical representation of the number of Iba-1 positive cells per mm² are illustrated in FIG. 4D.

As illustrated in FIGS. 4C and 4D, it was confirmed that the number of Iba-1 positive activated microglia was significantly increased by treatment with LPS and that the number of microglia activated by the PTP inhibitor was decreased. In particular, the PTP1B inhibitor (PTP1Bi) and the RPTPβ inhibitor (RPTPβi) decrease the activation of LPS-induced microglia in the hippocampus and cortex, the TC-PTP inhibitor (TC-PTPi) in the cortex, and the SHP2 inhibitor (SHP2i) in the hippocampus.

As shown by the above experimental results, it was confirmed that the PTP inhibitor in vitro and in vivo has an effect of reducing the activation of microglia under inflammatory conditions.

Additional experiments were performed on PTP1B among various types of PTPs.

### Example 4. Identification of the Increase in NO Production by the Overexpression of PTP1B in LPS-Stimulated Microglia

From the above examples, it was confirmed that the expression of PTP1B in microglia was increased under inflammatory conditions, and in order to confirm the role of increased PTP1B expression in terms of function, the following experiment was conducted. The BV2 microglial cell line (HA-PTP1B) stably overexpressing HA-PTP1B prepared by being transformed into the HA-PTP1B plasmid was prepared to confirm the enhanced PTP1B expression in the cell line prepared by Western blotting. The results are illustrated in FIG. 5.

As illustrated in FIG. 5, the expression level of PTP1B was increased more than twice in the BV2 microglial cell line (HA-PTP1B) overexpressing PTP1B compared to the naive BV2 microglial cell line, confirming that the cell line was appropriately prepared. They were used in the following examples.

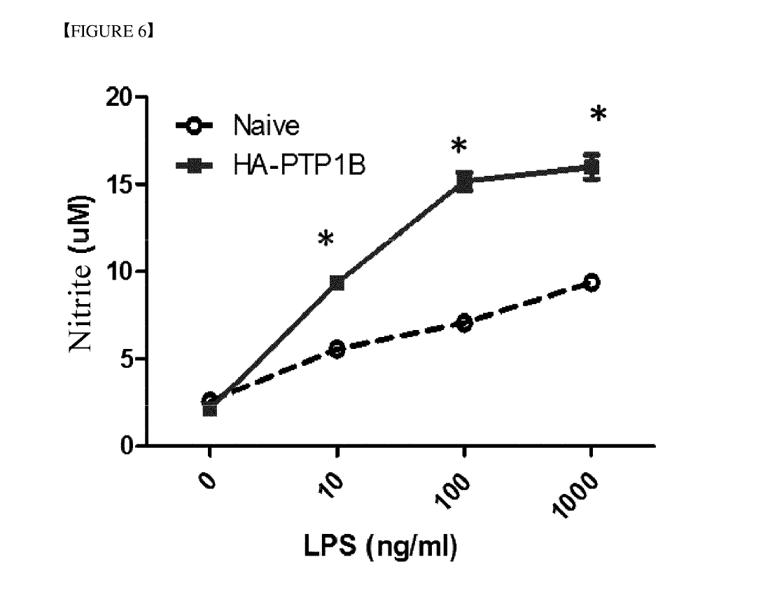

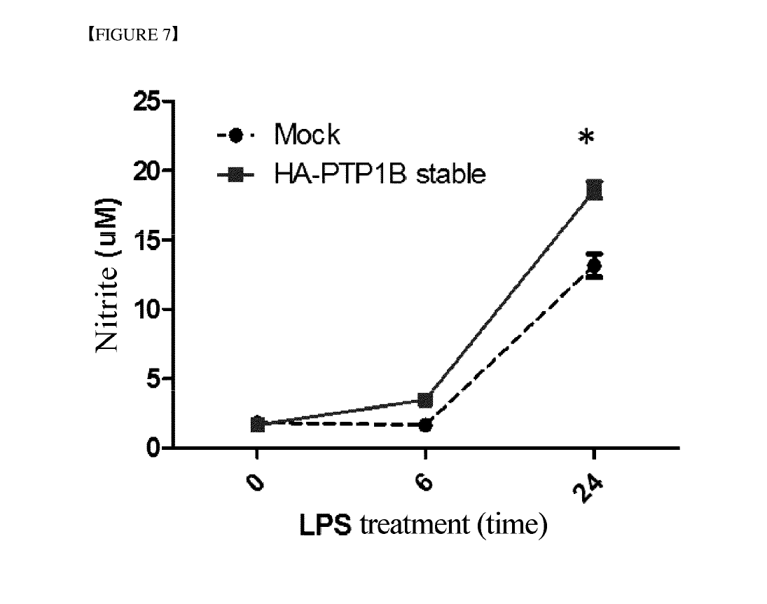

Since the production of NO (nitric oxide) is a strong marker of inflammatory response in microglia, the effect of PTP1B on LPS-induced NO production was investigated through the Griess reaction. The production of NO was measured using the amount of nitrite. Naive microglia or microglia overexpressing PTP1B were treated with LPS (originated from E. coli 055: B5; Sigma). FIG. 6 illustrates the result of treatment with a BV2 microglial cell line at the indicated dose of LPS. FIG. 7 illustrates the results of treating a BV2 microglial cell line with 100 ng LPS at the indicated time. 24 hours after incubation, 5 μl the cell culture media was mixed with Griess reagent with the same volume (0.1% naphthylethylenediamine dihydrochloride and 1% sulfanylamine in 5% phosphoric acid) in a 96-well microtiter plate. Absorbance was read at 540 nm and sodium nitrite was used for standard curve generation.

As illustrated in FIG. 6, it was confirmed that the NO production induced by LPS was dose-dependently increased for LPS, and NO accumulation was observed over time as illustrated in FIG. 7. That is, as for BV2 cells overexpressing PTP1B compared to naive BV2 cells, it was confirmed that LPS-induced NO production was increased over time dependently on the concentration of LPS.

### Example 5. Identification of Increase in the Expression of Proinflammatory Medium by PTP1B Overexpression in Microglia

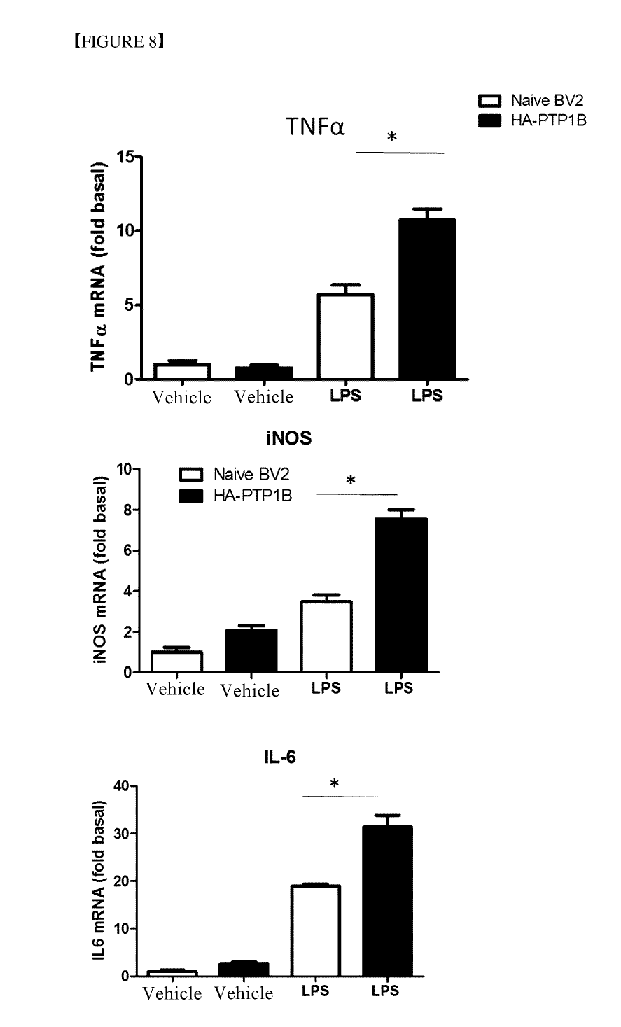

Real-time PCR was performed to confirm the effect of PTP1B on inflammatory cytokines, since the level of increased inflammatory cytokine is one of the markers showing the hyperactivation of microglia. Specifically, mRNA expression levels of inflammatory cytokines TNFα, iNOS and IL-6 in LPS-treated cells after treating naive BV2 cells and BV2 cells overexpressing PTP1B with LPS were measured by real-time PCR, and the results are illustrated in FIG. 8.

As illustrated in FIG. 8, it was confirmed that LPS-induced expression of TNFα, iNOS and IL-6 was increased in BV2 cells overexpressing PTP1B as compared with naive cells in the control group.

In other words, PTP1B induces the expression of inflammatory cytokines in microglia, indicating hyperactivation of microglia, so that it can be inferred that microglia are hyperactivated by PTP1B.

### Example 6. Identification of the Inhibition of Proinflammatory Signal-Induced NO Production by PTP1B Inhibitor in Microglia

As confirmed in Example 5 above, the overexpression of PTP1B increases the production of NO and proinflammatory cytokines under inflammatory conditions. The present inventors assumed from this result that the inhibition of PTP1B would prevent hyperactivation of microglia. In order to demonstrate this hypothesis, a PTP1B inhibitor ((S)-4-(((S)-1-(12-azanyl)-3-(4-(difluoro(phosphono)methyl)phenyl)-1-oxopropan-2-yl)amino)-3-((S)-3-(4-(difluoro(phosphono)methyl)phenyl)-2-pentadecanamidopropanamido)-4-oxobutanoic acid) was obtained from Dr. Zhang group and was used. The PTP1B inhibitor (hereinafter referred to as “iPTP1B”) used in the present invention was proved to be highly specific to PTP1B.

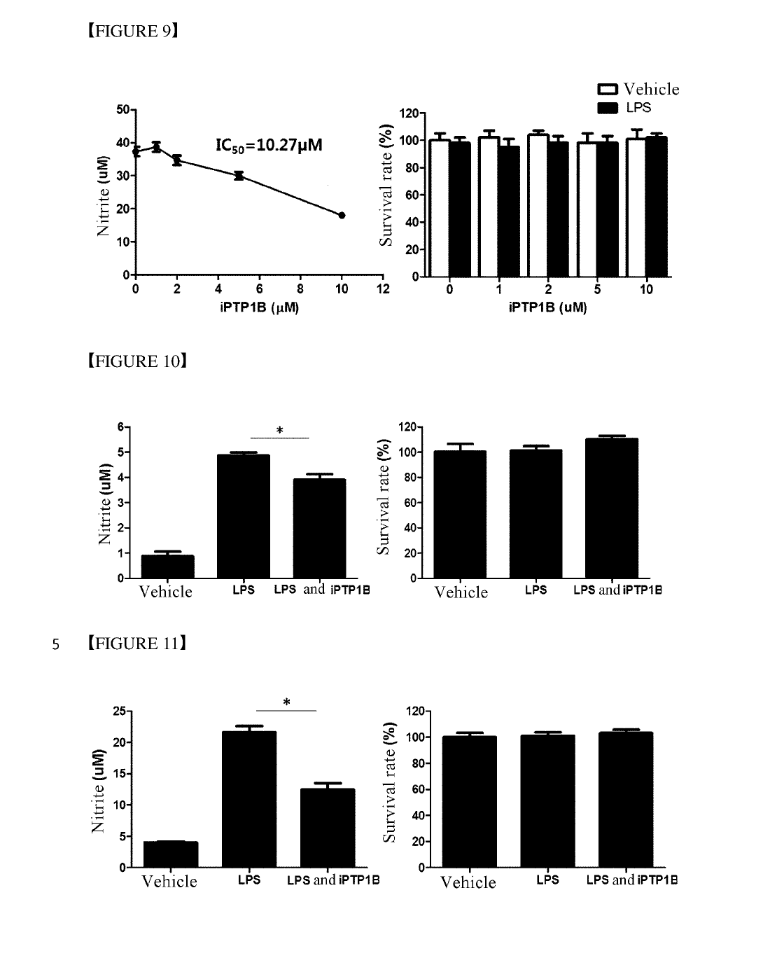

The effect of iPTP1B on the production of NO in LPS-induced BV2 microglia was examined in order to confirm the inflammatory inhibition effect of iPTP1B. The BV2 microglial cell lines were pretreated with the indicated different concentrations of iPTP1B and stimulated with LPS (100 ng/ml) 1 hour later, and the NO levels were measured according to the Griess method in the treated cells. In order to confirm cytotoxicity of iPTP1B in microglia, MTT assay was performed 24 hours after iPTP1B treatment, and the results are illustrated in FIG. 9.

As illustrated in FIG. 9, the level of LPS-induced NO in the microglia was dose-dependently decreased by iPTP1B and the IC50 value was found to be 10.27 μM. In addition, no significant level of cytotoxicity of iPTP1B was observed at the concentrations used in the test.

In addition, the treatment with iPTP1B alone did not inhibit or increase NO production, and significantly inhibited the level of NO inducing overproduction by LPS, suggesting that iPTP1B itself does not change the basic level of NO production.

In addition, the effect of iPTP1B on NO production was confirmed in mouse primary microglia. Specifically, primary microglia were pre-treated with 5 μM iPTP1B and then stimulated with LPS (50 ng/ml) for 24 hours. NO levels were confirmed in the stimulated primary microglia. In order to confirm the cytotoxicity of iPTP1B in primary microglia, MTT assay was performed 24 hours after iPTP1B treatment, and the results are illustrated in FIG. 10.

As illustrated in FIG. 10, it was confirmed that the level of LPS-induced NO was significantly decreased by iPTP1B. In addition, no significant level of cytotoxicity of iPTP1B was observed at the concentrations used in the test.

In addition, the effect of iPTP1B on NO production was confirmed in HAPI cells, a rat microglial cell line. Specifically, a rat microglial cell line, HAPI cells, were pre-treated with 10 μM iPTP1B for 1 hour and then stimulated with LPS (100 ng/ml) for 24 hours. NO levels were confirmed in the stimulated rat microglial cell line, HAPI cells. In order to confirm the cytotoxicity of iPTP1B in HAPI cells, a rat microglial cell line, MTT assay was performed 24 hours after iPTP1B treatment, and the results are illustrated in FIG. 11.

As illustrated in FIG. 11, it was confirmed that the level of LPS-induced NO was significantly decreased by iPTP1B. In addition, no significant level of cytotoxicity of iPTP1B was observed at the concentrations used in the test.

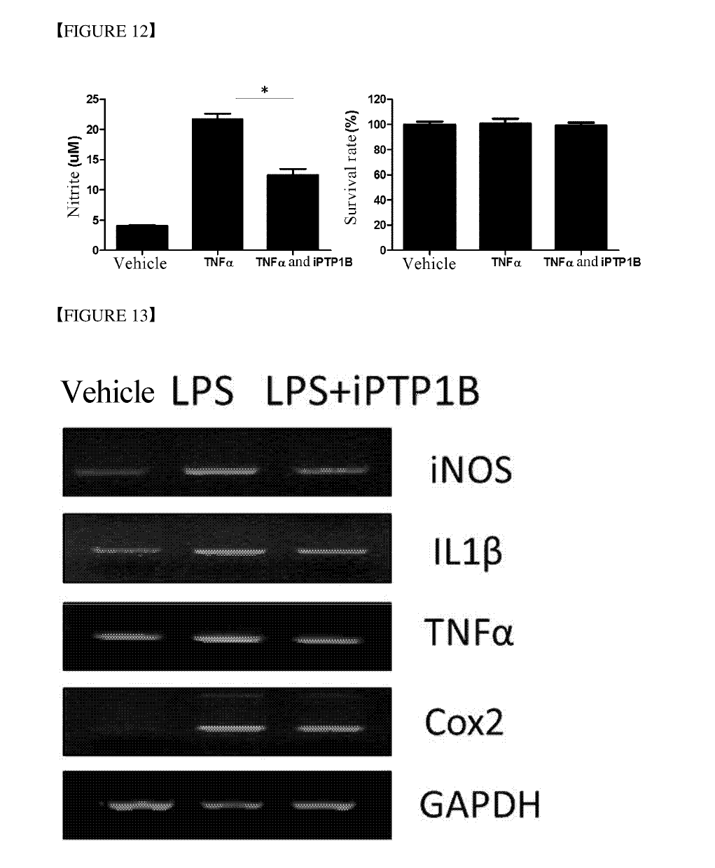

In addition, in order to confirm that TNFα-induced NO production was also inhibited by iPTP1B, HAPI cells, a rat microglial cell line, were pre-treated with 10 μM iPTP1B for 1 hour and then stimulated with TNFα (100 ng/ml) for 24 hours. NO levels were identified in the stimulated cells. In addition, in order to confirm the cytotoxicity of iPTP1B in HAPI cells, a rat microglial cell line, MTT assay was performed 24 hours after iPTP1B treatment, and the results are illustrated in FIG. 12.

As illustrated in FIG. 12, it was confirmed that the TNFα-induced NO level was significantly decreased by iPTP1B. In addition, no significant level of cytotoxicity of iPTP1B was observed at the concentrations used in the test.

Therefore, it was confirmed that LPS-induced NO production can be inhibited by PTP1B inhibitors in BV2 microglia, primary microglia, and rat HAPI microglia. It was also confirmed that TNFα-induced NO production in HAPI microglia could be inhibited by PTP1B inhibitors.

### Example 7. Identification that PTP1B Inhibitors Regulate the Production of LPS-Induced Proinflammatory Mediators

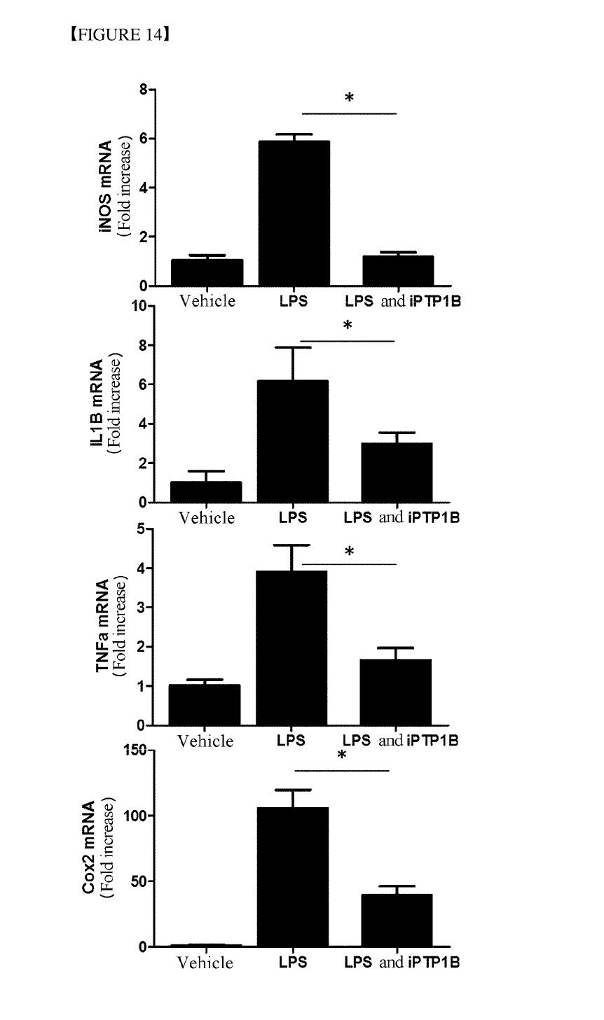

In order to confirm whether PTP1B inhibitors regulate the production of LPS-induced proinflammatory mediators, the effect of iPTP1B on the production of proinflammatory cytokines in BV2 microglia was determined. Specifically, BV2 microglia were treated with LPS (100 ng/ml) for 6 hours under the presence or absence of 10 μM iPTP1B, and mRNA expression levels of proinflammatory molecules iNOS, IL1β, TNFα, and Cox2 in the treated cell samples were measured by RT-PCR. The results are illustrated in FIG. 13. The results of the band intensity obtained by RT-PCR quantified and displayed in a graph are illustrated in FIG. 14.

As illustrated in FIGS. 13 and 14, pretreatment of iPBP1B significantly inhibits the production of LPS-induced cytokines and proinflammatory molecules (iNOS, IL1β, TNFα, and Cox2).

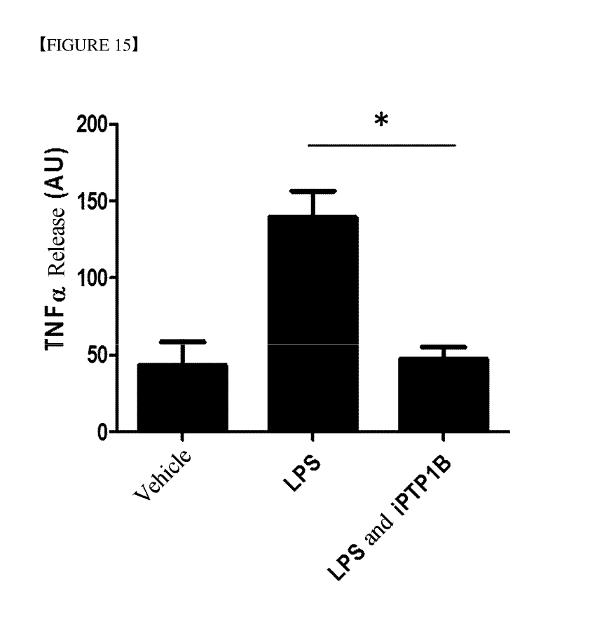

In addition, the level of TNFα protein in the BV2 microglial culture media treated with LPS as described above was confirmed by ELISA. Specifically, BV2 cells were treated with LPS in the presence or absence of iPTP1B. After 24-hour incubation, the TNFα levels in the culture media were measured using a rat monoclonal anti-mouse TNFα antibody as the capture antibody and a goat biotinylated polyclonal anti-mouse TNFα antibody as the detection antibody. The measured levels of TNFα protein results are illustrated in FIG. 15.

As illustrated in FIG. 15, when iPTP1B was treated, the expression level of TNFα in the culture media was significantly low, confirming that the PTP1B inhibitor inhibited the release of LPS-induced TNFα.

Accordingly, the treatment with iPTP1B on BV2 microglia inducing inflammation by LPS inhibited the production of proinflammatory factors iNOS, IL1β, TNFα, and Cox2, thus confirming that an PTP1B inhibitor inhibits inflammation.

### Example 8. Identification of Src as a Target Molecule of PTP1B in Microbial Activity

The above examples confirmed that PTP1B increases the neuroinflammatory response and the following was performed to confirm how PTP1B increases the LPS-induced inflammatory response. Based on the literature search, Src kinase, tyrosine kinase, among other known PTP1B substrates were selected as the target of PTP1B. The reason for this selection is that Src has a negative regulatory phosphorylation site (Y527). PTP1B can dephosphorylate the negative regulatory site of Src, which induces Src kinase activity.

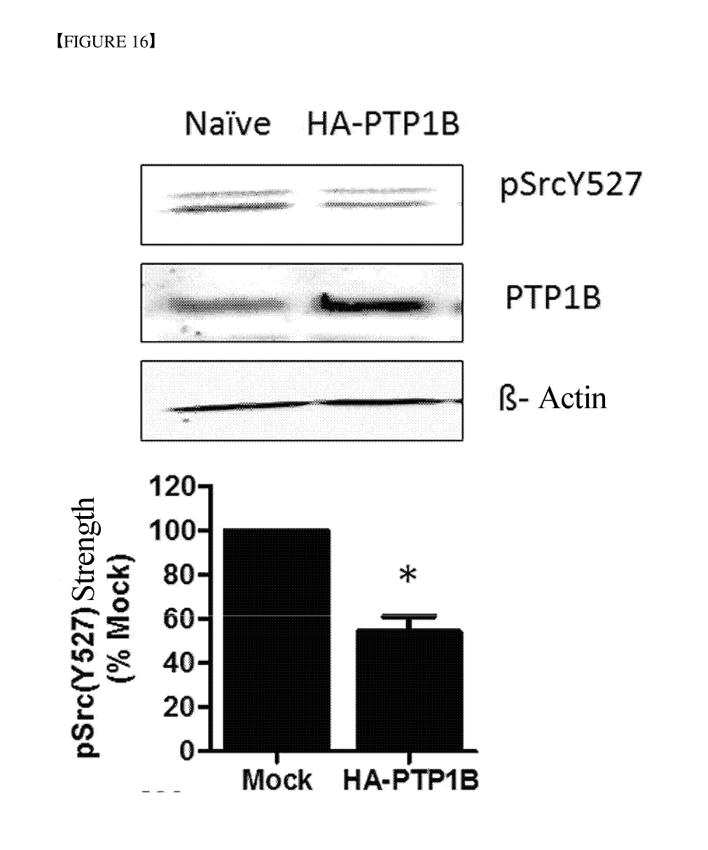

In order to confirm whether PTP1B is able to dephosphorylate Src and thereby activate Src in microglia, BV2 microglia were transfected with HA-PTP1B to produce BV2 microglia overexpressing PTP1B. The results of comparing the phosphorylation of Src at Y527 between BV2 microglia overexpressing PTP1B produced as such and naive BV2 microglia, and the results of the band intensity normalized and quantified with beta-actin and illustrated in a graph are illustrated in FIG. 16.

As illustrated in FIG. 16, it was confirmed that the phosphorylation of Src at Y527 was reduced up to 60% in the PTP1B-overexpressed microglia compared to the control group. This is consistent with previous research results and indicates that PTP1B acts to dephosphorylate of Src at Y527.

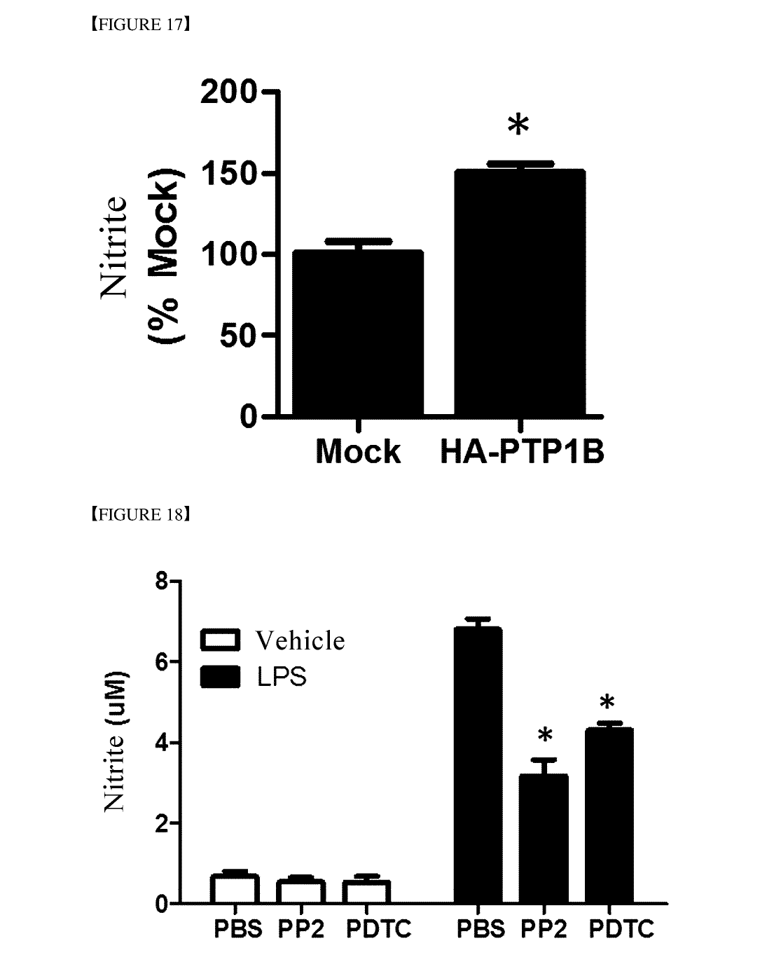

In addition, changes in LPS-induced NO production in microglial cell lines overexpressing PTP1B were confirmed, and the results are illustrated in FIG. 17.

As illustrated in FIG. 17, it was confirmed that LPS-induced NO levels were increased in PTP1B-overexpressed microglial cell lines, indicating that PTP1B overexpression, which increases Src activity, increased NO levels.

In addition, the level of LPS-induced NO production after BV2 treatment with Src kinase inhibitor PP2 (5 μM) or PDTC (Ammonium pyrrolidinedithiocarbamate, NFκB inhibitor) was identified to determine whether Src is associated with LPS-induced microglia activity. This is illustrated in FIG. 18.

As illustrated in FIG. 18, it was confirmed that the Src kinase PP2 inhibited significantly LPS-induced NO production in microglia as much as the IKK inhibitor, PDTC, and it was confirmed that inhibition of LPS-induced NO production by PP2 pretreatment was dose-dependent.

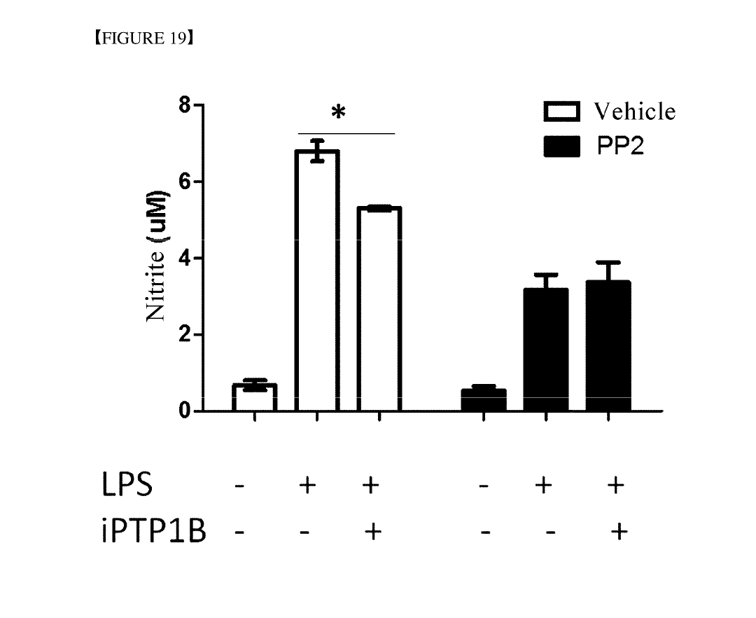

Next, it was determined whether the PTP1B-mediated proinflammatory response was dependent on Src activity in microglia. For this, BV2 microglia pretreated with Src inhibitor PP2 or iPTP1B for 1 hour were treated with LPS for 24 hours. NO levels were measured in the treated BV2 microglia and the results are illustrated in FIG. 19. The anti-inflammatory effect of iPTP1B was investigated.

As illustrated in FIG. 19, it was confirmed that PP2 treatment eliminated the anti-inflammatory effect of iPTP1B in microglia. These data demonstrate that PTP1B-mediated microglial activation is dependent on Src activity.

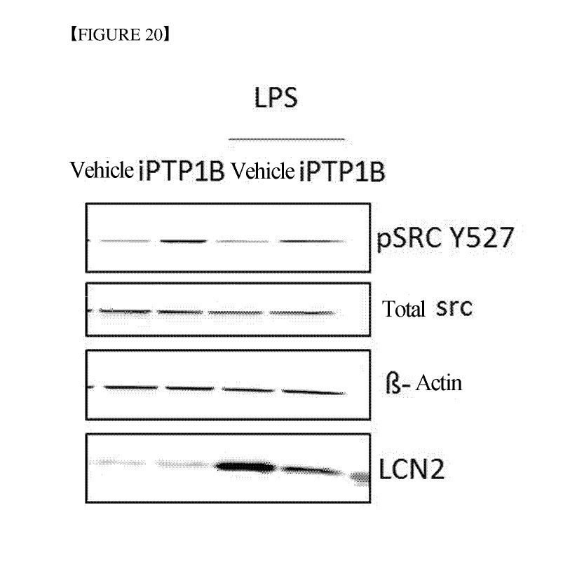

In addition, in vivo experiments were carried out using an inflammatory mouse model injected with LPS to identify the effect of PTP1B on phosphorylation of Src at Y527 in vivo. LPS+iPTP1B or iPTP1B was injected into the brain and phosphorylation of Src at Y527 was confirmed after 24 hours. At this time, total Src and beta-actin were used as a loading control group and Lnc2 was used as a marker of neuroinflammation. The results are illustrated in FIG. 20.

As illustrated in FIG. 20, it was confirmed that PTP1B activity inhibited by the injection of iPTP1B increased phosphorylation of Src at Y527. It was confirmed therefrom that increased levels of PTP1B in microglia can improve the signal transduction of inflammatory cytokines and that PTP1B can act as a proinflammatory factor via dephosphorylation of Src at Y527 in microglia.

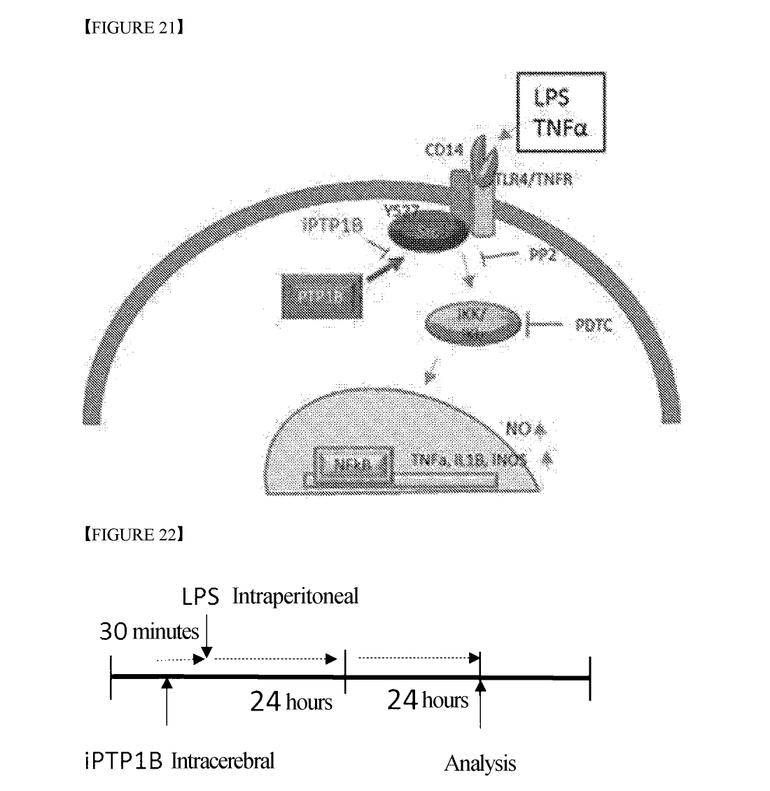

In other words, PTP1B promotes the production of proinflammatory cytokines and activates Src through dephosphorylation at Y527. This activated Src activates NFκB and increases the expression of proinflammatory factors. A schematic diagram of such a mechanism is briefly illustrated in FIG. 21.

### Example 9. Identification that the PTP1B Inhibitor Limits Microglia-Mediated Neuroinflammation In Vivo

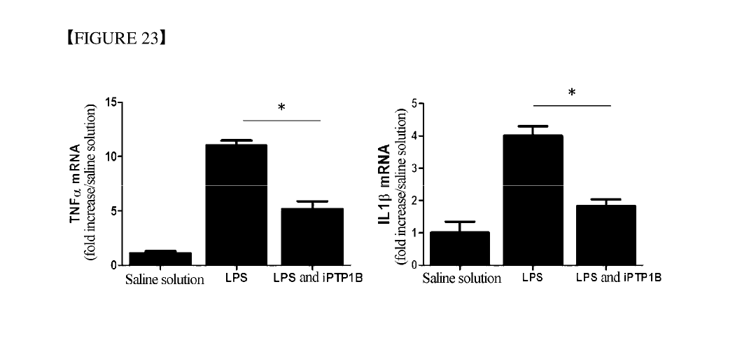

In order to confirm the anti-inflammatory effect of iPTP1B in vivo, the production of proinflammatory factors after LPS and iPTP1B injection in brain tissues was measured. The expression level of TNFα and IL1β genes, which are proinflammatory factors, was measured by real-time PCR 6 hours after the injection of LPS, and the results are illustrated in FIG. 23.

As illustrated in FIG. 23, the expressions of TNFα and IL1β were significantly weakened by inhibition of PTP1B in the inflammatory brain.

Accordingly, inflammatory stimuli increased PTP1B expression to induce microglial hyperactivation in the brain. Inhibiting PTP1B activity under inflammatory conditions prevented microglial hyperactivation in vitro and in vivo. Thus, it can be understood that the use of PTP inhibitors can effectively prevent or treat neuroinflammatory diseases, particularly inflammatory diseases in the brain.

Although the present invention has been described in terms of the preferred embodiments mentioned above, it is possible to make various modifications and variations without departing from the spirit and scope of the invention. It is also to be understood that the appended claims are intended to cover such modifications and variations as falling within the scope of the invention.

### Preparation Example 1. Manufacture of Medicines

1.1. Manufacture of Powder

PTP inhibitor 1-15 mg/L

Lactose 100 mg

Talc 10 mg

The above components are mixed and packed in airtight bags to prepare powders.

1.2. Manufacture of Tablets

PTP inhibitor 1-15 mg/L

Corn starch 100 mg

Lactose 100 mg

Magnesium stearate 2 mg

After mixing the above components, tablets are prepared by tableting according to a conventional method for producing tablets.

1.3. Manufacture of Capsules

PTP inhibitor 1-15 mg/L

Corn starch 100 mg

Lactose 100 mg

Magnesium stearate 2 mg

The above components are mixed according to a conventional method for producing capsules and filled in gelatin capsules to produce tablets.

1.4. Manufacture of Injections

PTP inhibitor 1-15 mg/L

Sterile distilled water for injection—suitable amount

pH adjuster—suitable amount

Injections are produced with the above component contents per 1 ampoule (2 ml) in accordance with a convention method for producing injections.

1.5. Manufacture of Liquid Agents

PTP inhibitor 1-15 mg/L

Sugar 20 g

Isomerized glucose syrup 20 g

Lemon flavor—suitable amount

Purified water was added to adjust the total volume to 1,000 ml. The above components are mixed in accordance with a conventional method for producing liquid agents, and then filled in a brown bottle and sterilized to produce liquid agents.

### Preparation Example 2. Manufacturing of Food Products

PTP inhibitor 1-15 mg/L

Vitamin mixture—suitable amount

Vitamin A acetate 70 μg

Vitamin E 1.0 mg

Vitamin B1 0.13 mg

Vitamin B2 0.15 mg

Vitamin B6 0.5 mg

Vitamin B 12 0.2 μg

Vitamin C 10 mg

Biotin 10 μg

Nicotinic acid amide 1.7 mg

Folic acid 50 μg

Calcium pantothenate 0.5 mg

Mineral mixture—suitable amount

Ferrous sulfate 1.75 mg

Zinc oxide 0.82 mg

Potassium monophosphate 15 mg

Calcium phosphate dibasic 55 mg

Potassium citrate 90 mg

Calcium carbonate 100 mg

Magnesium chloride 24.8 mg

Although the composition ratio of the above vitamin and mineral mixture is comparatively mixed with components suitable for a health functional food as a preferred embodiment, the compounding ratio may be arbitrarily changed and performed, and the above components may be mixed in accordance with a conventional method for producing health functional food, and then used in the manufacture of a health functional food composition (for example, nutritional candy, etc.) according to a conventional method.

### Preparation Example 3. Manufacturing of Beverages

PTP inhibitor 1-15 mg/L

Citric acid 1,000 mg

Oligosaccharide 100 g

Plum concentrate 2 g

Taurine 1 g

Purified water was added to obtain a total of 900 ml The above components were mixed according to a conventional method for producing health functional beverage, and the mixture was stirred and heated at 85° C. for about 1 hour. The resulting solution was filtered to obtain a sterilized 2 l container, sealed sterilized and refrigerated. Then, it is used for the production of the health functional beverage composition of the present invention.

Although the composition ratio was mixed and constructed with the components suitable for a relatively favorite beverage as a preferred embodiment, it is also possible to arbitrarily modify the compounding ratio according to the regional and national preferences such as the demand level, the demand country, and the purpose of uses.

1. A pharmaceutical composition for prevention or treatment of a neuroinflammatory disease, the pharmaceutical composition comprising a PTP (protein tyrosine phosphatase) inhibitor.

2. The pharmaceutical composition for prevention or treatment of a neuroinflammatory disease according to claim 1, wherein the PTP inhibitor is an inhibitor of one or more PTPs selected from the group consisting of PTP1B (Protein tyrosine phosphatase type 1B), TC-PTP (T-cell phosphatase), SHP2 (Src homology domain2-containing PTP2), MEG2 (Megakaryocyte-PTP2), LYP (Lymphoid specific-tyrosine phosphatase) and RPTPβ (Receptor-type tyrosine protein phosphatase beta).

3. The pharmaceutical composition for prevention or treatment of a neuroinflammatory disease according to claim 1, wherein the PTP inhibitor includes a compound described in the following table or a pharmaceutically acceptable salt thereof.

| Names of Compounds | Target PTP |
|---|---|
| (S)-4-(((S)-1- | PTP1B |
| (l2-azanyl)-3-(4-(difluoro(phosphono)methyl)phenyl)-1-oxopropan- | |
| 2-yl)amino)-3-((S)-3-(4-(difluoro(phosphono)methyl)phenyl)-2- | |
| pentadecanamidopropanamido)-4-oxobutanoic acid | |
| ((4-((S)-3-(((S)-1-amino-6-(4-ethylbenzamido)- | TC-PTP |
| 1-oxohexan-2-yl)amino)-2-((S)-2-(2-(((1R,2R,5S)-2-isopropyl- | |
| 5-methylcyclohexyl)oxy)acetamido)-3-phenylpropanamido)-3- | |
| oxopropyl)phenyl)difluoromethyl)phosphonic acid | |
| ((4- | SHP2 |
| ((S)-3-(((S)-1-(((S)-1-amino-3-(2-(4-hydroxy-3-methoxyphenyl)acetamido)- | |
| 1-oxopropan-2-yl)amino)-5-(3-iodobenzamido)-1-oxopentan-2- | |
| yl)amino)-6-hydroxy-3-iodo-1-methyl-2-(3-(2-oxo-2-((4-thiophen-3- | |
| yl)phenyl)amino)acetamido)phenyl)-1H-indole-5-carboxylic acid | |
| ((4-((S)-3-(((S)-1-(((S)-1-amino-3-(2-(4-hydroxy- | MEG2 |
| 3-methoxyphenyl)acetamido)-1-oxopropan-2-yl)amino)-5-(3-iodobenzamido)- | |
| 1-oxopentan-2-yl)amino)-2-(3-bromo-4-methylbenzamido)- | |
| 3-oxopropyl)phenyl)difluoromethyl)phosphonic acid | |
| 3-((3-Chlorophenyl)ethynyl)- | LYP |
| 2-(4-(2-(cyclopropylamino)-2-oxoethoxy)phenyl)-6-hydroxybenzofuran- | |
| 5-carboxylic Acid | |
| 2-(3-(2-(3-bromo-5-iodobenzamido)acetamido)phenyl)- | RPTPβ |
| 6-hydroxy-3-iodo-1-methyl-1H-indole- | |
| 5-carboxylic acid | |

4. The pharmaceutical composition for prevention or treatment of a neuroinflammatory disease according to claim 1, wherein the neuroinflammatory disease is selected from the group consisting of multiple sclerosis, neuroblastoma, stroke, Alzheimer's disease, Parkinson's disease, Lou Gehrig's disease, Huntington's disease, Creutzfeldt Jakob disease, post-traumatic stress disorder, depression, schizophrenia, and amyotrophic lateral sclerosis.

5. The pharmaceutical composition for prevention or treatment of a neuroinflammatory disease according to claim 1, wherein the neuroinflammatory disease is a brain inflammatory disease.

6. The pharmaceutical composition for prevention or treatment of a neuroinflammatory disease according to claim 1, wherein the PTP inhibitor inhibits neuroinflammation through inhibition of activation of microglia.

7. The pharmaceutical composition for prevention or treatment of a neuroinflammatory disease according to claim 1, wherein the PTP inhibitor exhibits an inhibitory effect on neuroinflammation by reducing Src activity.

8. A food composition for improving a neuroinflammatory disease, the food composition comprising a PTP inhibitor.
